(19) United States
(12) Patent Application Publication
(10) Pub. No.: US 2020/0297488 A1
(43) Pub. Date: Sep. 24, 2020

(54) A PROSTHETIC DEVICE FOR MITRAL VALVE REPAIR

(71) Applicant: I BIRDI LTD, LONDON (GB)

(72) Inventor: INDERPAUL BIRDI, LONDON (GB)

(21) Appl. No.: 16/089,823

(22) PCT Filed: Mar. 31, 2017
(86) PCT No.: PCT/GB2017/050925

§ 371 Date: Sep. 28, 2018

(30) Foreign Application Priority Data
Mar. 31, 2016 (GB) 1605510.5

(51) Int. Cl. A61F 2/24

(52) U.S. Cl. CPC A61F 2/2448; A61F 2250/0029; A61F 2250/0018

(57) ABSTRACT

A prosthetic ring or band (700) for repair of a mitral valve is disclosed, having a substantially annular form or a part annular form, wherein the prosthetic ring or band occupies at least a part of a shape having: preferably, a substantially straight base portion (710), configured to align with the anterior leaflet of the mitral valve; and a central curved portion (720), arranged opposite the base portion, having a first radius of curvature, Ri, and a first and second lateral curved portions located either side of the central curved portion; wherein the first and second lateral curved portions have a second radius of curvature, R₂; wherein R₂>Ri; and wherein at least a portion of one of the first or second lateral curved portions is removed. Related methods of preparation of such rings or bands and repair of mitral valves in human or animal hearts with such rings or bands are also disclosed.

### TECHNICAL FIELD

The present application relates to a prosthetic for mitral valve repair, methods of use and manufacture of a prosthetic for mitral valve repair in a human or animal heart.

### BACKGROUND TO THE INVENTION AND PRIOR ART

The mitral valve (also commonly known as the bicuspid valve or left atrioventricular valve) is one of the four valves that control blood flow within the human heart. The mitral valve is the valve which controls blood flow between the left atrium and the left ventricle of the heart. As the blood pressure increases within the left atrium (from blood arriving in the pulmonary veins), the mitral valve opens to allow blood to flow from the left atrium into the left ventricle. Subsequently, the mitral valve closes upon contraction of the heart to prevent blood flowing back into the left atrium. Hence, the mitral valve is commonly known as the “in-flow valve” of the heart.

As can be imagined, malfunction of the mitral valve can have serious health effects. There are many well-known causes of mitral valve malfunction, including congenital conditions, degenerative conditions and disease. The background causes of malfunction of the mitral valve, in so far as they are understood, are known in the art, and so are not explained in detail in the present disclosure. The present invention relates to methods and apparatus for rectification of the defects in the mitral valve once they have been detected.

FIG. 1 provides a schematic illustration of a typical human heart. Two of the heart's valves are illustrated in FIG. 1, the mitral valve 100 and the tricuspid valve 150. In the present disclosure, only the mitral valve 100 will be further discussed. The mitral valve 100 is made up of two “flaps” 110, 120 which, when the valve 100 is closed, closely align to prevent blood flow through the valve. A healthy valve 110 has an approximately 3:4 anteroposterior diameter 130 to transverse diameter 140 ratio during systole (the contraction phase of the cardiac cycle). I.e. mitral heart valves 110 are generally annular, or “kidney-shaped”, valves. FIGS. 2 and 3 are respective cross-section representations of healthy 200 and un-healthy 300 human mitral valves during systole. The mitral valve 200 shown in FIG. 2 has the correct geometric ratio and hence the two flaps 210 and 220 closely align during systole. The mitral valve 200 is made up of two flaps known as the anterior leaflet 210 and the posterior leaflet 220. The boundaries between the anterior leaflet 210 and the posterior leaflet 220 are marked by the anterolateral commissure line 230 and the posteromedial commissure line 240.

The mitral valve 200 is typically further divided, according to Carpentier's surgical classification, as follows. The mitral valve posterior leaflet 220 is divided into three scallops, the anterior scallop 221, the middle scallop 222 and the posterior scallop 223. The mitral valve anterior leaflet 210 is divided into three corresponding segments, the anterior segment 211, the middle segment 212 and the posterior segment 213. Finally, two further segments are defined on the mitral valve anterior leaflet 210, the anterior commissure 270 and the posterior commissure 280.

In the healthy mitral valve 200, it can be seen that corresponding scallops (221, 222, 223) and segments (211, 212, 213) closely align during systole, forming a seal. FIG. 3 shows a mitral valve with an abnormal geometric ratio, wherein it can be seen that corresponding scallops (321, 322, 323) and segments (311, 312, 313) no longer align, leaving gap 390.

The mitral heart valve shown in FIG. 3 has an abnormal geometric ratio, causing the two flaps 310, 320 fail to align properly. This can lead to an insufficient seal of the mitral valve 300, which in turn leads to blood regurgitation and reverse blood flow.

Mitral heart valves 300 with abnormal geometric ratios are commonly fixed by the suturing of a prosthetic ring to the mitral valve which remodels its shape, by altering the anteroposterior and transverse diameters, back to the geometric ratio of a normally functioning valve. In cases where these prosthetic rings have been surgically installed, normal blood flow through the heart and the pumping function of the heart can usually be restored.

The surgical techniques necessary to install mitral valve prosthetics are collectively known in the art as mitral valve annuloplasty. Generally, these techniques involve isolating the heart from the circulatory system, accessing the left atrium, aligning the mitral valve prosthetic and then stitching (fixing) the mitral valve prosthetic onto the mitral valve to effect a permanent re-orientation of the valve geometric ratio.

The use of mitral valve prosthetic rings is generally considered to have been pioneered by the French heart surgeon Alain Carpentier. Since the creation and use of the first mitral valve prosthetic in the 1980s many designs of mitral valve prosthetics have been created and used by surgeons, some of which are shown in FIGS. 4A to 4F, as will be described in more detail in the following sections.

Whilst suitable surgical techniques have been established, considerable debate remains over the best form of mitral valve prosthetic to use, in particular due to the natural variation in anatomy throughout the population. Prior art mitral valve prosthetics generally conform to the annular kidney shape of the mitral valve they are designed to repair.

As can be appreciated from FIGS. 4A to 4F, differences in the exact shape are known in the prior art. However, fundamentally, the shapes of mitral valve prosthetics are restricted to those that conform, at least partially, to the shape of a mitral heart valve. As shown in FIGS. 4A to 4F, many different form factors of mitral valve prosthetic have been developed. However, there has been a consistent belief that the posterior leaflet must in all cases be supported around its outer edge, by any prosthesis used in the prior art. Despite the availability of an extensive body of optional arrangements of mitral valve prosthetics, post-surgical complications can still be experienced by patients who have undergone mitral valve repair.

A typical mitral valve prosthetic 500 is shown in FIG. 5. The prosthetic 500 can be divided into four sections. The prosthetic 500 has a substantially straight base portion 510, a central curved portion 520 located opposite the base portion 510, and two lateral curved portions 530, 540 located between the base portion 510 and the curved portion 550. Thus, the prosthetic 500 forms a substantially annular “kidney shaped” form.

FIG. 6a shows a cross section of a human heart 600 in which a mitral valve 690 has been repaired by the fixation of a prior art mitral valve prosthetic 650. In FIG. 6a the mitral valve has been marked where possible in accordance with the convention of FIG. 2, that is the anterior leaflet 610, the posterior leaflet 620 are indicated.

The mitral valve prosthetic 650 has previously been defined as divided into four sections. The mitral valve prosthetic 650 has a substantially straight base portion 651, a central curved portion 652 located opposite the base portion 651, and two lateral curved portions 653, 654 located between the base portion 651 and the curved portion 652.

FIG. 6a illustrates the close shape alignment of the mitral valve prosthetic 650 to the mitral valve. Consequently, shape of the mitral valve prosthetic 650 can be further defined, for ease of reference, in relation to the anatomical definitions of the mitral valve. The substantially straight base portion 651 closely aligns to the aortic mitral curtain. The central curved portion 652 and two lateral curved portions 653, 654 closely align with the perimeter of the mitral valve posterior leaflet 620.

The mitral valve 690 and the mitral valve prosthetic 650 can be further defined. FIG. 6b shows an enlarged version of mitral valve 690 and the mitral valve prosthetic 650, in which they further definitions are added. The markings of FIG. 6b should be understood as applicable to all comparable figures in the present application. The mitral valve 690 can be further divided, according to Carpentier's surgical classification. The mitral valve posterior leaflet 620 is divided into three scallops, the anterior scallop 621, the middle scallop 622 and the posterior scallop 623. The mitral valve anterior leaflet 610 is divided into three corresponding segments, the anterior segment 611, the middle segment 612 and the posterior segment 613. Finally, two further segments are defined on the mitral valve anterior leaflet 610, the anterior commissure 670 and the posterior commissure 680.

Moreover, the central curved portion 652 and two lateral curved portions 653, 654 of the mitral valve prosthetic can be even further defined in relation to Carpentier's surgical classification of the mitral valve. The central curved portion 651 and two lateral curved portions 653, 654 of the mitral valve prosthetic can be divided into five “zones” 655 to 659. Zone 655 corresponds to the anterior commissure 670, zone 656 corresponds to the anterior scallop 621, zone 657 corresponds to the middle scallop 622, zone 658 corresponds to the posterior scallop 623, and zone 659 corresponds to the posterior commissure 680.

Mitral valve repair is generally successful, with some studies estimating that there is up to a 95% chance that no further surgical intervention will be required in the 10 years following a mitral valve annuloplasty (Heart. 2006 February; 92(2): 275-281). However, despite the low re-operation rate of mitral valve annuloplasties, there remains a risk of causing damage to the tissue surrounding the mitral valve prosthesis upon fixation of the device.

Despite the low re-operation rate, it is typically expected that approximately 20% of patients will suffer some degree of reduced ventricular function following a mitral valve repair annuloplasty. Of this 20% of patients, up to a third do not fully their pre-operation ventricular function.

### SUMMARY OF THE INVENTION

Other features and advantages of the present invention will be apparent from the appended claims.

In a first aspect, the present invention provides a prosthetic ring for repair of a mitral valve, the prosthetic ring forming at least part of a shape having: a substantially straight base portion, configured to align with the aortic mitral curtain; a central curved portion, arranged opposite the base portion, having a first radius of curvature, R₁; and a first and second lateral curved portions, located either side of the central curved portion, wherein the first and second lateral curved portions have a second radius of curvature, R₂; wherein R₂<R₁; and a gap in the circumference of the ring formed in at least a portion of the first lateral curved portion or the second lateral curved portion.

In a second aspect, the present invention provides a prosthetic ring for repair of a mitral valve, having a substantially annular form or a part annular form, wherein the prosthetic ring occupies at least a part of a shape having: a substantially straight base portion, configured to align with the aortic mitral curtain; a central curved portion, arranged opposite the base portion, configured to align with the posterior leaflet of the mitral valve in the region of the middle scallop; a first lateral curved portion, located between the first end of the central curved portion and the first end of the base portion, configured to align with the anterolateral commissure of the mitral valve; a second lateral curved portion, located between the second end of the central curved portion and the second end of the base portion, configured to align with the posteromedial commissure of the mitral valve; and a gap in the circumference of the ring formed in at least a portion of the first lateral curved portion or the second lateral curved portion.

The prosthetic ring may further comprise a posterior ring part and an anterior ring part coupled to the posterior ring part at at least one coupling point on a transverse plane defining a maximum width of the prosthetic ring; wherein the prosthetic ring comprises a gap or a removable portion located in the posterior ring part.

The gap in the circumference of the prosthetic ring of the present invention may correspond to at least the portion of the prosthetic ring aligned with the anterolateral commissure. The gap in the circumference of the prosthetic ring of the present invention may also correspond to at least the portion of the prosthetic ring aligned with the anterior scallop of the mitral valve. Similarly, the gap of the prosthetic ring of the present invention may correspond to at least the portion of the prosthetic ring aligned with the circumflex branch of the left coronary artery.

A portion of the prosthetic ring of the present invention opposite a gap in the prosthetic ring may a) have an increased stiffness, b) have an increased thickness, or c) be formed from a stiffer material, than the rest of the prosthetic ring. Furthermore, the prosthetic ring of the present invention may comprise multiple layers of material. A portion of the prosthetic ring of the present invention opposite a gap in the prosthetic ring may have an increased number of layers of material. A portion of the prosthetic ring of the present invention opposite a gap in the prosthetic ring may be made of a material with a flexural modulus which is higher than the material of the remainder of a circumference of the prosthetic ring. Moreover, the prosthetic ring of the present invention may further comprise a suture permeable coating.

In a third aspect, the present invention provides a prosthetic ring for repair of a mitral valve, having a substantially annular form or a part annular form, wherein the prosthetic ring occupies at least a part of a shape having: a substantially straight base portion, configured to align with the aortic mitral curtain; a central curved portion, arranged opposite the base portion, configured to align with the posterior leaflet of the mitral valve in the region of the middle scallop; a first lateral curved portion, located between the first end of the central curved portion and the first end of the base portion, configured to align with the anterolateral commissure of the mitral valve; a second lateral curved portion, located between the second end of the central curved portion and the second end of the base portion, configured to align with the posteromedial commissure of the mitral valve; and wherein the prosthetic ring comprises a cut-able portion, located on the first lateral curved portion or the second lateral curved portion, which when removed, provides a gap in the circumference of the ring formed in the first lateral curved portion or the second lateral curved portion.

In a fourth aspect, the present invention provides a method of preparing a prosthetic ring for repair of a mitral valve, comprising: removing a portion of the ring; wherein the prosthetic ring occupies at least a part of a shape having: a substantially straight base portion, configured to align with the aortic mitral curtain; a central curved portion, arranged opposite the base portion, configured to align with the posterior leaflet of the mitral valve in the region of the middle scallop; a first lateral curved portion, located between the first end of the central curved portion and the first end of the base portion, configured to align with the anterolateral commissure of the mitral valve; a second lateral curved portion, located between the second end of the central curved portion and the second end of the base portion, configured to align with the posteromedial commissure of the mitral valve; and wherein removing the portion of the ring provides a gap in the circumference of the ring formed in the first lateral curved portion or the second lateral curved portion.

The method of preparing the prosthetic ring may be performed on a prosthetic ring having a substantially annular form or a part annular form, the prosthetic ring further comprising a posterior ring part and an anterior ring part coupled to the posterior ring part at at least one coupling point on a transverse plane defining a maximum width of the prosthetic ring; wherein removing the portion of the ring to provide a gap in the circumference of the ring comprises forming a gap located in the posterior ring part.

The method of preparing the prosthetic ring may further comprise forming a gap in at least the portion of the prosthetic ring configured to align with the anterolateral commissure of the mitral valve. The method of preparing the prosthetic ring may further comprise forming a gap in at least the portion of the prosthetic ring configured to align with the anterior scallop of the mitral valve. The method of preparing the prosthetic ring may further comprise forming a gap in at least the portion of the prosthetic ring configured to align with the circumflex branch of the left coronary artery.

In a fifth aspect, the present invention provides a prosthetic band for repair of a mitral valve, the prosthetic band forming at least part of a shape having: a central curved portion, having a first radius of curvature, R₁; and a first and second lateral curved portions, located either side of the central curved portion, wherein the first and second lateral curved portions have a second radius of curvature, R₂; wherein R₂<R₁; and a gap in the band, the gap extending over a, preferably substantially straight, base portion configured to align with the aortic mitral curtain and at least a portion of the first lateral curved portion or the second lateral curved portion.

In a sixth aspect, the present invention provides a prosthetic band for repair of a mitral valve, having a substantially part annular form, wherein the prosthetic band occupies at least a part of a shape having: a central curved portion, configured to align with the posterior leaflet of the mitral valve in the region of the middle scallop; a first lateral curved portion, located at the first end of the central curved portion, configured to align with the anterolateral commissure of the mitral valve; a second lateral curved portion, located at the second end of the central curved portion, configured to align with the posteromedial commissure of the mitral valve; and a gap in the band, the gap extending over a, preferably substantially straight, base portion configured to align with the aortic mitral curtain and at least a portion of the first lateral curved portion or the second lateral curved portion.

The gap in the prosthetic band may extend over at least the portion of the prosthetic band configured to align with the anterolateral commissure of the mitral valve. The gap in the prosthetic band may also extend over at least the portion of the prosthetic band configured to align with the anterior scallop of the mitral valve. The gap in the prosthetic band may also extend over to at least the portion of the prosthetic band configured to align with the circumflex branch of the left coronary artery.

In a seventh aspect, the present invention provides a prosthetic band for repair of a mitral valve, having a substantially part annular form, wherein the prosthetic band occupies at least a part of a shape having: a central curved portion, configured to align with the posterior leaflet of the mitral valve in the region of the middle scallop; a first lateral curved portion, located at the first end of the central curved portion, configured to align with the anterolateral commissure of the mitral valve; a second lateral curved portion, located at the second end of the central curved portion, configured to align with the posteromedial commissure of the mitral valve; and wherein the prosthetic band comprises a cut-able portion, located on the first lateral curved portion or the second lateral curved portion, which when removed, provides a gap in the band extending over the first lateral curved portion or the second lateral curved portion.

In an eighth aspect, the present invention provides a method of preparing a prosthetic band for repair of a mitral valve, comprising: removing a portion of the prosthetic band; wherein the prosthetic band occupies at least a part of a shape having: a central curved portion, configured to align with the posterior leaflet of the mitral valve in the region of the middle scallop; a first lateral curved portion, located at the first end of the central curved portion, configured to align with the anterolateral commissure of the mitral valve; a second lateral curved portion, located at the second end of the central curved portion, configured to align with the posteromedial commissure of the mitral valve; and wherein removing the portion of the band provides a gap in the band extending over the first lateral curved portion or the second lateral curved portion.

Removing the portion of the band to provide a gap in the band may comprise forming a gap in at least the portion of the prosthetic band configured to align with the anterolateral commissure of the mitral valve. Removing the portion of the band to provide a gap in the band may also comprise forming a gap in at least the portion of the prosthetic band configured to align with the anterior scallop of the mitral valve. Removing the portion of the band to provide a gap in the band may also comprise forming a gap in at least the portion of the prosthetic band configured to align with the circumflex branch of the left coronary artery.

In a ninth aspect, the present invention provides a method of repairing a mitral valve using a prosthetic ring or prosthetic band according to any of the first to eight aspects, comprising the steps of: suturing the prosthetic ring, or the prosthetic band, to a mitral valve to remodel its shape.

In a tenth aspect, the present invention provides a method of repairing a mitral valve comprising the steps of: preparing a prosthetic ring or prosthetic band according to the method of any of the first to eighth aspects; and suturing the prosthetic ring, or the prosthetic band, to a mitral valve to remodel its shape.

It will be evident to the skilled reader that the above definitions of features of the invention relate to same or similar products and uses for such products and so any or all of the above described features of the invention can be separated or combined in various combinations while still realising the benefits of the invention.

Other features and advantages of the present invention will be apparent from the appended claims.

### BRIEF DESCRIPTION OF THE FIGURES

Some embodiments of apparatus and/or methods in accordance with embodiments of the present invention are now described, by way of example only, and with reference to the accompanying drawings, in which:

FIG. 1 shows a cross-section representation of a human heart

FIG. 2 shows a cross-section representation of a healthy human mitral valve during systole;

FIG. 3 shows a cross-section representation a misaligning mitral valve during systole;

FIGS. 4A to 4F show plan views of mitral valve prosthetics known from the prior art;

FIG. 5 shows a plan view of a mitral valve prosthetic known from the prior art;

FIG. 6a shows a cross-section representation of mitral valve repaired by fixation of a prior art mitral valve prosthetic;

FIG. 6b shows an enlarged representation of the repaired mitral valve of FIG. 6a;

FIG. 7 shows a mitral valve prosthetic according to the present invention;

FIG. 8 shows a cross-section representation of mitral valve repaired by fixation of a mitral valve prosthetic according to an embodiment of the present invention;

FIG. 9a shows a mitral valve prosthetic according to the present invention;

FIG. 9b shows a mitral valve prosthetic according to the present invention;

FIG. 10 shows a mitral valve prosthetic according to the present invention.

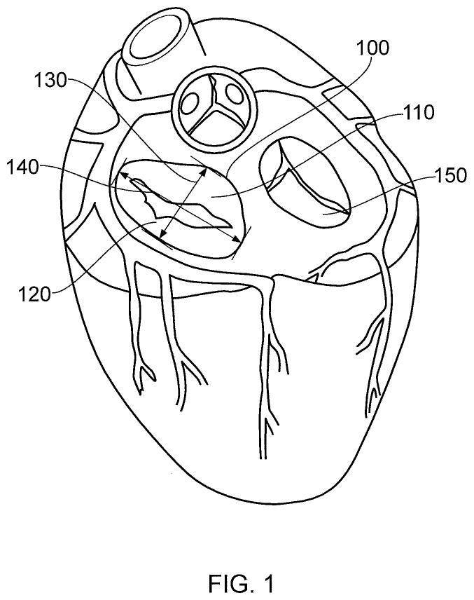

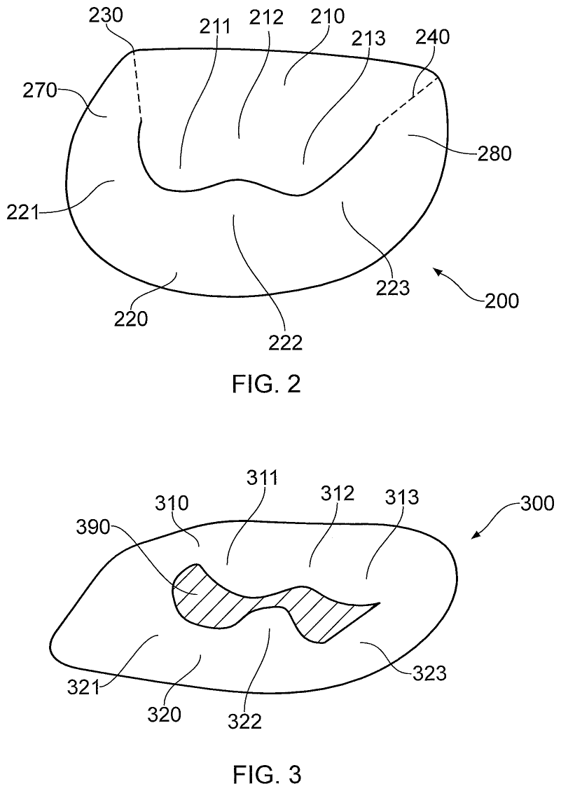

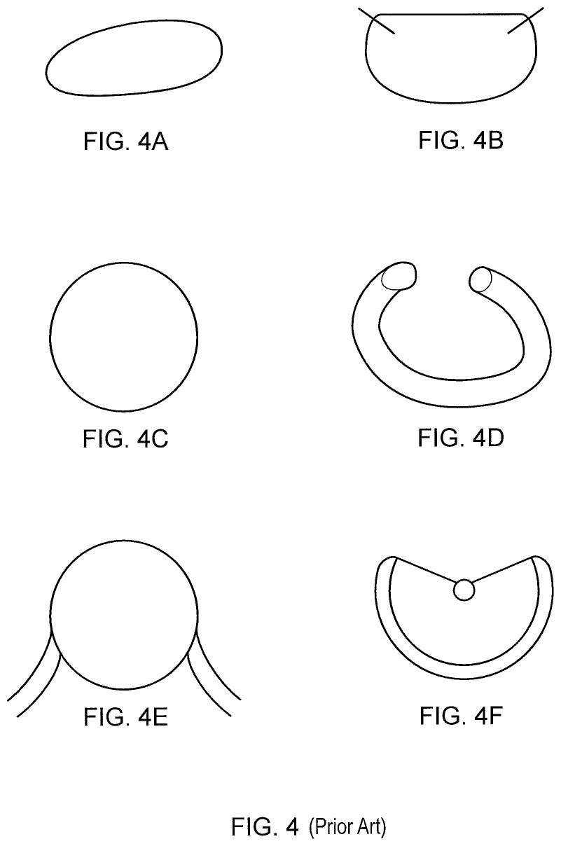

### DETAILED DESCRIPTION OF EMBODIMENTS

To aid the skilled person's understanding, where appropriate, an imaginary x-axis and y-axis has been included in the figures. Where included, the x-axis is marked “x”, and the y-axis is marked “y”.

The applicant has identified that 3% to 5% of patients will suffer some degree of circumflex artery distortion or injury, during a mitral valve annuloplasty. Circumflex artery damage may lead to a myocardial infarction (a heart attack), generally due to unrecoverable left ventricular infarction, unless quickly diagnosed and treated.

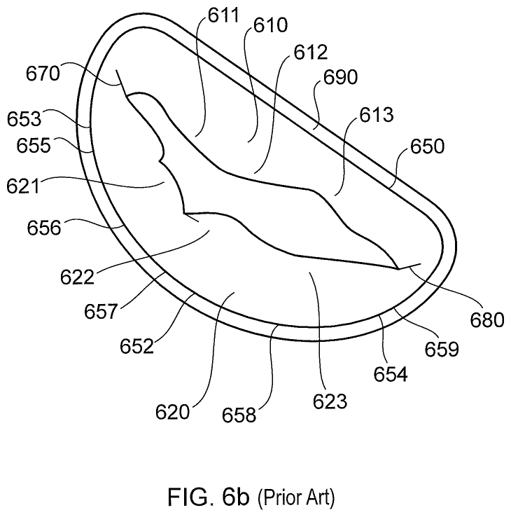

One difficulty faced by surgeons performing mitral heart valve annuloplasties is that the surgeon may not know beforehand the relative location of the heart vasculature to the operation site. The surface of the heart is covered by a complicated vasculature system, as can be seen, for example, in FIG. 6a.

The applicant has also identified that a significant potential cause of complications during or after mitral valve annuloplasty can be the surgical impingement of the vasculature in proximity to the mitral valve. These problems are particularly acute in patients who, through natural anatomical variation, are considered to have a higher risk anatomy. In particular, the applicant has identified that patients with heart vasculature in which the circumflex branch of the coronary artery lies close to the mitral valve are particularly at risk of operative complications during and following a mitral valve annuloplasty. Therefore, there exists a need for an improved mitral valve prosthetic which mitigates some of the causes of complications experienced in mitral valve annuloplasty procedures for those patients where natural variation has caused particularly high risk location of the vasculature in proximity of the mitral valve. Particularly, it has been identified that certain patients can be identified as at a high risk of operative circumflex artery damage.

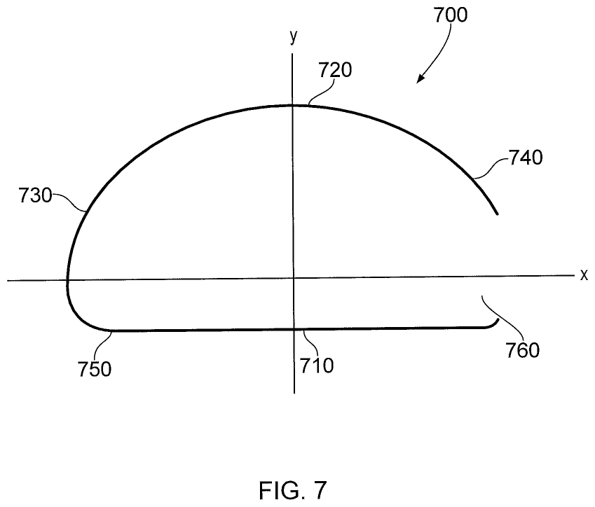

A first embodiment of the invention will now be described with reference to FIG. 7. FIG. 7 shows a mitral valve prosthetic 700. The shape of the prosthetic 700 conforms to an annular, kidney-shaped form. Thus, the shape of the prosthetic 700 can be divided into four sections. The shape of prosthetic 700 has a substantially straight base portion 710, a central curved portion 720 located opposite the base portion 710, and two lateral curved portions 730, 740 located between the base portion 710 and the curved portion 720.

Mitral valve prosthetic 700 may be made of a resilient bio-compatible material. The resilience of the material allows the prosthetic 700 to be at least partially deformed during surgery and provides a force to the mitral valve which restores the natural anteroposterior diameter to transverse diameter ratio geometric ratio. Bio-compatibility ensures that the prosthetic 700 does not promote blood clotting or bacterial growth. Many suitable resilient bio-compatible materials are known in the art, including bio-compatible polymers, metals and metal-alloys. Preferably, at least part of the prosthetic 700 is made of titanium or a titanium alloy.

The mitral valve prosthetic 700 may be covered in suture-permeable covering 750. The suture-permeable covering can aid the surgeon in fixing the prosthetic 700 to the mitral valve. Suture-permeable coverings for prosthetics are well known in the art and the prosthetic 700 may use any such suitable covering. Preferably, the suture-permeable covering is Dacron® (polyethylene terephthalate) coated silicon.

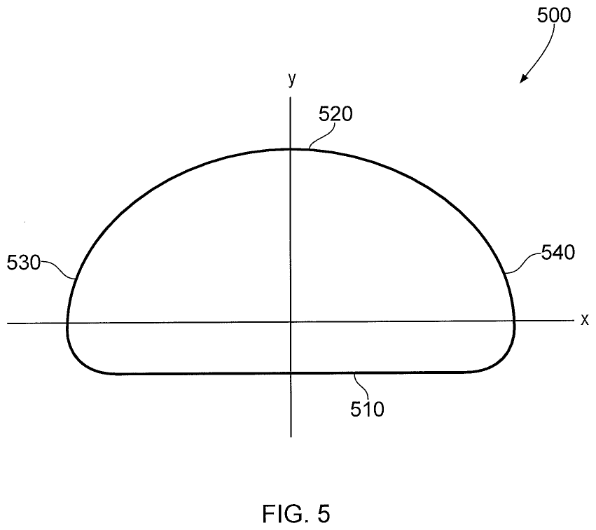

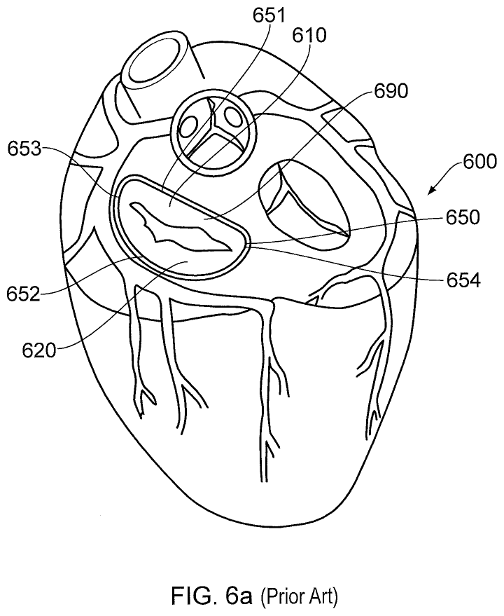

The mitral valve prosthetic 700 has a gap 760 in its circumference. The gap 760 corresponds to the region of the mitral valve close to the circumflex artery and represents either a manufactured gap or removed portion in the circumference of the prosthetic. Since gap 760 is provided in the prosthetic 700, in use, no sutures can be placed within the region of the mitral valve closest to the circumflex artery. This reduces the likelihood of circumflex artery impingement or damage. This goes against the strongly held prejudice in the art to provide as much support as possible to the circumference of the mitral valve; or in cases where the anterior leaflet of the mitral valve is unaffected by the underlying pathology, to provide as much support as possible to at least the entirety of the posterior leaflet of the mitral valve. Evidence of this prejudice in the art can be seen, as discussed previously, in prior art prostheses such as those shown in FIGS. 4A to 4F.

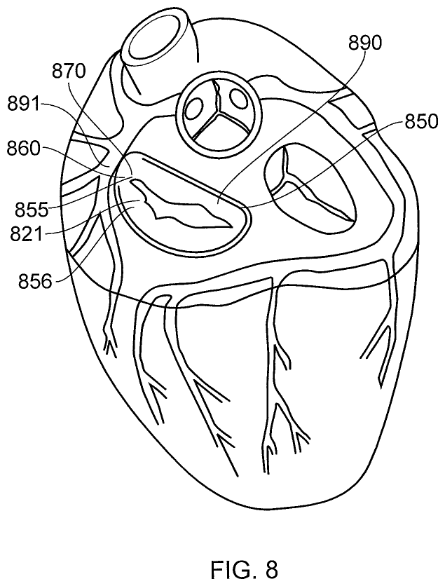

FIG. 8 illustrates the mitral valve prosthetic 850 once it has been fixed to a mitral valve 890. As can be seen from FIG. 8, parts of the circumflex artery 891 (otherwise known as the circumflex branch of the coronary artery) are located close to the mitral valve 890. Therefore, the gap 760, 860 corresponds to, at least, the anterior commissure 670, 870, i.e. zone 655, 855. Furthermore, FIG. 8 also illustrates that the region of the circumflex artery 891 in close proximity to the mitral valve 890 may extend from the region of the anterior commissure 670, 870 to at least partially surround the region of the anterior scallop 621, 821. Consequently, the gap 760, 860 may also correspond to, at least part of, the anterior commissure 670, 870 and the anterior scallop 621, 821 i.e. zone 655, 855 and zone 656, 856.

As will be appreciated, the gap 760, 860 in the mitral valve prosthetic 700 will reduce the overall stiffness of the mitral valve prosthetic 700. To compensate, the mitral valve prosthetic preferably comprises a material with a stiffness higher than a prior art mitral valve prosthetic. Increased stiffness can be achieved by any suitable method of stiffening an object, including using a material with a higher stiffness, combining multiple layers of material, thickening at least a portion of the ring and/or incorporating multiple materials of differing stiffnesses into the ring. This is preferably implemented in an area substantially opposite or adjacent to gap 760, 860.

The applicant recognises that omitting or removing a portion of the mitral valve prosthetic 700 may decrease the durability of the prosthetic 700 due to increased flexing under contraction of the heart. This is a further reason for which the configurations of the present invention have been avoided in the prior art. However, in conceiving the invention, it has been recognised that clinically, the potentially lower durability of the prosthetic 700 may be preferable to the risk of damage to heart vasculature through infarction of the lateral wall of the heart for certain patients due to damage caused to vasculature in close proximity to the area of suturing.

In FIG. 7, mitral valve prosthetic 700 is shown as being substantially symmetrical along a central midline. The skilled person will understand that the shape of mitral valve prosthetic 700 can deviate from being perfectly symmetrical whilst still conforming to the shape of the mitral valve. In fact, the mitral valve prosthetic must be at least partially deformable in order to be installed by common key-hole surgical methods. Furthermore, the mitral valve prosthetic is sutured onto a mitral valve which continuously deforms as the heart beats. Thus, in use, the mitral valve prosthetic 700 must resiliently correct the shape of the mitral valve whilst allowing for the natural cycle of movement of the valve.

FIG. 7 also illustrates only a 2-dimensional representation of mitral valve prosthetic 700. This should not be taken to mean that mitral valve prosthetic 700 is flat in the third dimension (i.e. a z-axis which is normal to both the marked x-axis and y-axis). In fact, mitral valve prosthetic 700 may be formed to have a substantially curved profile along the z-axis, as is used in some prior art mitral valve prosthetics.

Mitral valves come in many different sizes and consequently, the mitral valve prosthetic 700 may be supplied in a series of differing sizes, all conforming to the same shape of the claimed invention, to fit differing sizes of mitral valves. The larger mitral valve prosthetics 700 may require increased stiffening in order to sufficiently correct the abnormal geometric ratio of the damaged mitral valves. This increased stiffening may be performed by any of the methods described above. Conversely, the smaller sizes of mitral valve prosthetic may require less stiffening in order to adequately correct the mitral valve.

Whilst mitral valve prosthetics with removed sections are known from the prior art, generally termed “mitral bands” (see for example FIG. 4D of the present application), all of these prior art mitral valve prosthetics define a removed or removable section within the substantially straight base portion (710) of the mitral valve prosthetic; i.e. the prior art mitral valves with a gap are effectively “C” shaped. These “C” shaped mitral valves are so shaped in order to preserve the natural shape and function of the anterior leaflet of the mitral valve, whilst providing full support around the circumference of the posterior leaflet. Consequently, these prior art mitral valve prosthetics should only be used where the anterior leaflet of the mitral valve is unaffected by the underlying pathology requiring treatment. Thus, these prior art mitral valve prosthetics are aimed at solving a different technical problem to the presently claimed invention, that of providing a mitral valve prosthetic which does not alter or restrict the shape and movement of the anterior leaflet of the mitral valve. Differently to the present invention, all prior art mitral valve prosthetics have until now provided support to the whole of the posterior leaflet of the mitral valve.

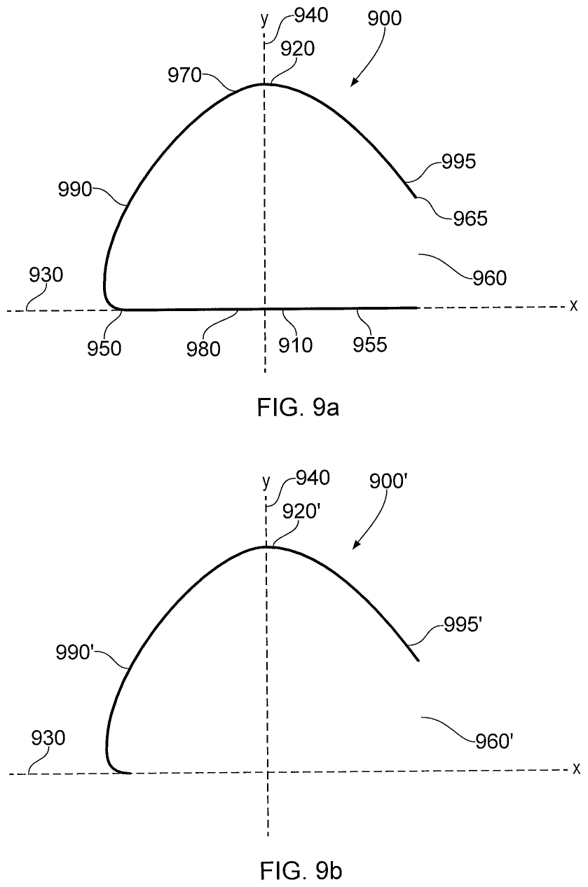

Further embodiments will now be further described in relation to FIG. 9a. The mitral valve prosthetic 900 conforms to mitral valve 700, shown in FIG. 7, but is defined geometrically rather than by reference to anatomical features. Mitral valve prosthetics are commonly described geometrically in the prior art, as can be seen in EP 1258232 A2 and U.S. 2011/0224786 A1. Mitral valve 900 is thus described, where possible, using equivalent geometric terms as those used in the prior art.

Mitral valve prosthetic 900 comprises an anterior half-ring 910 which is substantially straight, and a curved posterior half-ring 920. The posterior half-ring 920 and anterior half-ring 910 are coupled at one point 950 located substantially on a lateral plane 930 that defines a maximum width section of the prosthesis. In addition, a longitudinal plane 940 is also defined, that intersects the prosthesis substantially at points 970 and 980, that is arranged substantially perpendicular to the lateral plane 930 and substantially bisects the anterior half-ring 950 at point 980. The posterior half-ring 920 is thus subdivided in to a first lateral zone (left) 990 located between the points 950 and 970, and a second lateral zone (right) 995 located between the points 965 and 970.

The intersection points 950, 970 and 980 of the prosthesis respectively with the planes 930 and 940 define geometric points used for the calculation of the dimensions of the prosthesis. In accordance with the prior art, the distance between the points 970 and 980, defines the height of the prosthesis. The length of the anterior half-ring 910, defines a width of the prosthesis. Typically, the ratio of the height of the mitral valve prosthesis to the width of the prosthesis is approximately 3:4.

Mitral valve prosthetic 900 is made of a resilient bio-compatible material. The resilience of the material allows the prosthetic 900 to be at least partially deformed during surgery and provides a force to the mitral valve which restores the natural anteroposterior diameter to transverse diameter ratio geometric ratio. Bio-compatibility ensures that the prosthetic 900 does not promote blood clotting or bacterial growth. Many suitable resilient bio-compatible materials are known in the art, including bio-compatible polymers, metals and metal-alloys. Preferably, at least part of the prosthetic 900 is made of titanium or a titanium alloy.

The mitral valve prosthetic 900 is covered in suture-permeable covering 955. The suture-permeable covering aids the surgeon in fixing the prosthetic 900 to the mitral valve. Suture-permeable coverings for prosthetics are well known in the art and the prosthetic 900 may use any such suitable covering. Preferably, the suture-permeable covering is Dacron® (polyethylene terephthalate) coated silicon.

The mitral valve prosthetic 900 has a gap 960. The gap 960 is provided in, or omitted from, at least a portion of the first or second lateral curved portions 990 or 995. Since portion 960 is removed from the prosthetic 900, in use, no sutures can be placed within these regions. This portion 960 corresponds to the region of the mitral valve closest to the circumflex artery, thus reducing the likelihood of circumflex artery impingement or damage.

As will be appreciated, removing a portion 960 of the mitral valve prosthetic 900 will reduce the overall stiffness of the mitral valve prosthetic 900. To compensate, the mitral valve prosthetic is preferably made of a material with a stiffness higher than a prior art mitral valve prosthetic. Increased stiffness can be achieved by any prior art method of stiffening an object, including using a material with a higher stiffness, combining multiple layers of material, thickening at least a portion of the ring and/or incorporating multiple materials of differing stiffnesses into the ring. This is preferably implemented in an area substantially opposite or adjacent to gap 960.

The applicant recognises that omitting or removing a portion of the mitral valve prosthetic 900 may decrease the durability of the prosthetic 900 due to increased flexing under contraction of the heart. However, it has been recognised that clinically, the potentially lower durability of the prosthetic 900 may be preferable to the risk of damage to heart vasculature through infarction of the lateral wall for certain patients.

In FIG. 9a, mitral valve prosthetic 900 is shown as symmetrical along a central midline. The skilled person will understand that the shape of mitral valve prosthetic 900 can deviate from being perfectly symmetrical whilst still conforming to the shape of the mitral valve. In fact, the mitral valve prosthetic must be at least partially deformable in order to be installed by common key-hole surgical methods. Furthermore, the mitral valve prosthetic is sutured onto a mitral valve which continuously deforms as the heart beats. Thus, in use, the mitral valve prosthetic 900 must resiliently correct the shape of the mitral valve whilst allowing for the natural cycle of movement of the valve.

Mitral valves come in many different sizes and The mitral valve prosthetic 900 may be supplied in a series of differing sizes, all conforming to the same shape of the claimed invention, to fit differing sizes of mitral valves. The larger mitral valve prosthetics 900 may require increased stiffening in order to sufficiently correct the abnormal geometric ratio of the damaged mitral valves. This increased stiffening may be performed by any of the methods described above.

Further embodiments will now be described in relation to FIG. 9b. FIG. 9b illustrates the mitral valve prosthetic 900 of FIG. 9a in a prosthetic band configuration. As has been described previously, prosthetic bands (or “mitral bands”) are known in the art. These known prosthetic bands can comprise a removed or removable section within the previously defined substantially straight base portion (710) of the mitral valve prosthetic. These known prosthetic bands are shaped in order to preserve the natural shape and function of the anterior leaflet of the mitral valve. Therefore, these prosthetic bands are preferably only used when the anterior leaflet of the mitral valve is functionally normal and does not require further support.

There are patients who would otherwise be suitable for a prosthetic band, but have an anatomy which gives rise to a high risk of surgical impingement of the vasculature in proximity to the mitral valve. Hence, they are not suitable for traditional prosthetic bands. As will be appreciated from the discussion above, the presently described novel features of the invention can be equally applicable to a prosthetic band arrangement. For illustration, an example is shown in FIG. 9b. Thus, FIG. 9b shows a prosthetic band, in accordance with embodiments of the present invention, which are particularly suitable for patients with high risk anatomies, but a fully functioning anterior leaflet of the mitral valve.

As described earlier in relation to mitral bands, in FIG. 9b, mitral valve prosthetic 900′ does not comprise an anterior half-ring 910, but otherwise corresponds in shape to the mitral valve prosthetic 900 of FIG. 9a. That is, in FIG. 9b, mitral valve prosthetic 900′ comprises a curved posterior half-ring 920′. FIG. 9b also illustrates that mitral valve prosthetic 900′ comprises a gap 960′ which extends over the base portion (not shown), i.e. the missing anterior half-ring 910 of FIG. 9a.

As with the example of FIG. 9a, the mitral valve prosthetic 900′ of FIG. 9b is also made of a resilient bio-compatible material. The resilience of the material allows the prosthetic 900′ to be at least partially deformed during surgery and provides a force to the mitral valve which restores the natural anteroposterior diameter to transverse diameter ratio geometric ratio. Bio-compatibility ensures that the prosthetic 900′ does not promote blood clotting or bacterial growth. Many suitable resilient bio-compatible materials are known in the art, including bio- compatible polymers, metals and metal-alloys. Preferably, at least part of the prosthetic 900′ is made of titanium or a titanium alloy.

As with the example of FIG. 9a, the mitral valve prosthetic 900′ of FIG. 9b is also covered in suture-permeable covering (not shown). The suture-permeable covering aids the surgeon in fixing the prosthetic 900′ to the mitral valve. Suture-permeable coverings for prosthetics are well known in the art and the prosthetic 900′ may use any such suitable covering. Preferably, the suture-permeable covering is Dacron® (polyethylene terephthalate) coated silicon.

As with the example of FIG. 9a, the mitral valve prosthetic 900′ has a gap 960′. The gap 960′ is provided in, or omitted from, at least a portion of the first or second lateral curved portions 990′ or 995′. Since portion 960′ is removed from the prosthetic 900′, in use, no sutures can be placed within these regions. This portion 960′ corresponds to the region of the mitral valve closest to the circumflex artery, thus reducing the likelihood of circumflex artery impingement or damage.

As will be appreciated, the removed portion 960′ of the mitral valve prosthetic 900′ of FIG. 9b will further reduce the overall stiffness of the mitral valve prosthetic 900′. To compensate, the mitral valve prosthetic is preferably made of a material with a stiffness higher than a prior art mitral valve prosthetic. Increased stiffness can be achieved by any prior art method of stiffening an object, including using a material with a higher stiffness, combining multiple layers of material, thickening at least a portion of the ring and/or incorporating multiple materials of differing stiffnesses into the ring. This is preferably implemented in an area substantially opposite or adjacent to gap 960′ or the overall gap between ends of the band.

The applicant recognises that omitting or removing a greater portion of the mitral valve prosthetic 900′ may decrease the durability of the prosthetic 900′ due to increased flexing under contraction of the heart. However, it has been recognised that clinically, the potentially lower durability of the prosthetic 900′ may be preferable to the risk of damage to heart vasculature through infarction of the lateral wall for certain patients.

In FIG. 9b, mitral valve prosthetic 900′ is shown as substantially symmetrical along a central midline. The skilled person will understand that the shape of mitral valve prosthetic 900′ can deviate from being perfectly symmetrical whilst still conforming to the shape of the mitral valve. In fact, the mitral valve prosthetic must be at least partially deformable in order to be installed by common key-hole surgical methods. Furthermore, the mitral valve prosthetic is sutured onto a mitral valve which continuously deforms as the heart beats. Thus, in use, the mitral valve prosthetic 900′ must resiliently correct the shape of the mitral valve whilst allowing for the natural cycle of movement of the valve.

Mitral valves come in many different sizes and the mitral valve prosthetic 900′ may be supplied in a series of differing sizes, all conforming to the same shape of the claimed invention, to fit differing sizes of mitral valves. The larger mitral valve prosthetics 900′ may require increased stiffening in order to sufficiently correct the abnormal geometric ratio of the damaged mitral valves. This increased stiffening may be performed by any of the methods described above.

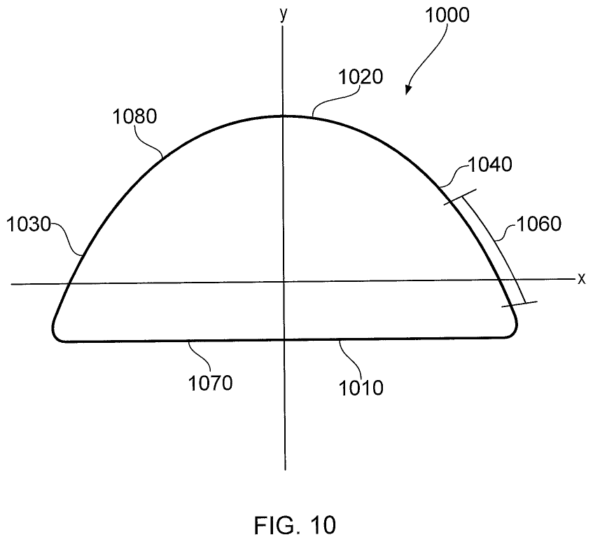

In a second embodiment of the present invention, with reference to FIG. 10, there is provided a mitral valve prosthetic 1000. The shape of the prosthetic 1000 conforms to the annular, kidney-shaped form of the prosthetic 700, 800 of the first embodiment. That is, the shape of the prosthetic 1000 can be divided into four sections; a substantially straight base portion 1010, a central curved portion 1020 located opposite the base portion 1010, and two lateral curved portions 1030, 1040 located between the base portion 1010 and the curved portion 1020. Alternatively, the shape of the prosthetic 1000 could be considered to comprise a posterior half-ring 1080 and an anterior half-ring 1070 which is coupled to the posterior half ring at at least one coupling point on a first lateral plane.

Mitral valve prosthetic 1000 may also be made of a resilient bio-compatible material. The resilience of the material allows the prosthetic 1000 to be at least partially deformed during surgery and provides a force to the mitral valve which restores the natural anteroposterior diameter to transverse diameter ratio geometric ratio. Bio-compatibility ensures that the prosthetic 1000 does not promote blood clotting or bacterial growth. Many suitable resilient bio-compatible materials are known in the art, including bio-compatible polymers, metals and metal-alloys. Preferably, at least part of the prosthetic 1000 is made of titanium or a titanium alloy.

The mitral valve prosthetic 1000 may also be covered in suture-permeable covering 1050. The suture-permeable covering can aid the surgeon in fixing the prosthetic 1000 to the mitral valve. Suture-permeable coverings for prosthetics are well known in the art and the prosthetic 1000 may use any such suitable covering. Preferably, the suture-permeable covering is Dacron® (polyethylene terephthalate) coated silicon.

The mitral valve prosthetic 1000 has a removable portion 1060. The removable portion 1060 corresponds to the region of the mitral valve close to the circumflex artery. The removable portion 1060 is weaker, and hence more easily cut-able, than the rest of the mitral valve prosthetic. I.e. the material making up the removable portion 1060 is less resistant to shear stresses than the material making up the rest of the mitral valve prosthetic. Any suitable method of providing a material less resistant to shear stress may be used, for example, using a different material in the removable portion 1060, using less layers of material in the removable portion 1060 and/or using thinner layers of material in the removable portion 1060.

The removable portion 1060 may be removed in use by the surgeon to provide a gap 1060 corresponding to the region of the mitral valve close to the circumflex artery. The removable portion, may be made in any portion of the posterior half-ring. The removable portion 1060 may be removed in its entirety or partially. When portion 1060 is removed from the prosthetic 1000, in use, no sutures can be placed within the region of the mitral valve closest to the circumflex artery, thus reducing the likelihood of circumflex artery impingement or damage. The removable portion 1060 may be made of a different material to the rest of the mitral valve prosthetic. Preferably, the removable portion 1060 is made of a material that is less tough than the rest of the ring, to aid the removal of the removable portion. The removable portion 1060 may further be segmented to aid its partial removal.

The suture-permeable covering 1050 may be marked to illustrate the extent of the removable portion 1060. Any bio-compatible marking may be used, such as changing the colouration of the suture-permeable covering, the addition of further bio-compatible threads.

As will be appreciated, removing a portion 1060 of the mitral valve prosthetic 1000 will reduce the overall stiffness of the mitral valve prosthetic 1000. To compensate, the mitral valve prosthetic preferably comprises a material with a stiffness higher than a prior art mitral valve prosthetic. Increased stiffness can be achieved by any prior art method of stiffening an object, including using a material with a higher stiffness, combining multiple layers of material, thickening at least a portion of the ring and/or incorporating multiple materials of differing stiffnesses into the ring.

The applicant recognises that removing a portion of the mitral valve prosthetic 1000 may decrease the durability of the prosthetic 1000 due to increased flexing under contraction of the heart. However, in conceiving the inventions, it has been recognised that clinically, the potentially lower durability of the prosthetic 1000 may be preferable to the risk of damage to heart vasculature through infarction of the lateral wall of the heart for some patients.

In FIG. 10, mitral valve prosthetic 1000 is shown as being substantially symmetrical along a central midline. The skilled person will understand that the shape of mitral valve prosthetic 1000 can deviate from being perfectly symmetrical whilst still conforming to the shape of the mitral valve. In fact, the mitral valve prosthetic must be at least partially deformable in order to be installed by common key-hole surgical methods. Furthermore, the mitral valve prosthetic is sutured onto a mitral valve which continuously deforms as the heart beats. Thus, in use, the mitral valve prosthetic 1000 must resiliently correct the shape of the mitral valve whilst allowing for the natural cycle of movement of the valve.

FIG. 10 also illustrates only a 2-dimensional representation of mitral valve prosthetic 700. This should not be taken to mean that mitral valve prosthetic 1000 is flat in the third dimension (z-axis). In fact, mitral valve prosthetic 1000 may be formed substantially curved along the z-axis, as is used in some prior art mitral valve prosthetics.

The removable portion 1060 can also be applied to the prosthetic band-type embodiment of FIG. 9b, to provide a removable portion at the location of the gap or omitted portion 960′. As can be seen from FIG. 9b, mitral valve prosthetic 900′ already comprises a gap 960′ which extends over the base portion (not shown), i.e. the missing anterior half-ring 910 of FIG. 9a. The removable portion 1060 extends gap 960′ over a portion of the first or second lateral curved portions 990′ or 995′.

Mitral valves come in many different sizes and consequently, the mitral valve prosthetic 1000 may be supplied in a series of different sizes, all conforming to the same shape of the claimed invention, to fit differing sizes of mitral valves. The larger mitral valve prosthetics 1000 may require increased stiffening in order to sufficiently correct the abnormal geometric ratio of the damaged mitral valves. This increased stiffening may be performed by any of the methods described above. Conversely, the smaller sizes of mitral valve prosthetic may require less stiffening in order to adequately correct the mitral valve.

1. A prosthetic ring for repair of a mitral valve, the prosthetic ring forming at least part of a shape having:
a substantially straight base portion, configured to align with the aortic mitral curtain;
a central curved portion, arranged opposite the base portion, having a first radius of curvature, R₁; and
a first and second lateral curved portions, located either side of the central curved portion, wherein the first and second lateral curved portions have a second radius of curvature, R₂;
wherein R₂<R₁; and
a gap in the circumference of the ring formed in at least a portion of the first lateral curved portion or the second lateral curved portion.

2. A prosthetic ring for repair of a mitral valve, having a substantially annular form or a part annular form, wherein the prosthetic ring occupies at least a part of a shape having:
a substantially straight base portion, configured to align with the aortic mitral curtain;
a central curved portion, arranged opposite the base portion, configured to align with the posterior leaflet of the mitral valve in the region of the middle scallop;
a first lateral curved portion, located between the first end of the central curved portion and the first end of the base portion, configured to align with the anterolateral commissure of the mitral valve;
a second lateral curved portion, located between the second end of the central curved portion and the second end of the base portion, configured to align with the posteromedial commissure of the mitral valve; and
a gap in the circumference of the ring formed in at least a portion of the first lateral curved portion or the second lateral curved portion.

3. The prosthetic ring of any of claims 1 to 2, wherein the prosthetic ring has a substantially annular form or a part annular form, and further comprises:
a posterior ring part;
an anterior ring part coupled to the posterior ring part at at least one coupling point on a transverse plane defining a maximum width of the prosthetic ring;
wherein the prosthetic ring comprises a gap or a removable portion located in the posterior ring part.

4. The prosthetic ring of any of claims 1 to 3, wherein the gap in the circumference of the prosthetic ring corresponds to at least the portion of the prosthetic ring configured to align with the anterolateral commissure of the mitral valve.

5. The prosthetic ring of any of claims 1 to 4, wherein the gap in the circumference of the prosthetic ring corresponds to at least the portion of the prosthetic ring configured to align with the anterior scallop of the mitral valve.

6. The prosthetic ring of any of claims 1 to 3, wherein the gap in the circumference of the prosthetic ring corresponds to at least the portion of the prosthetic ring configured to align with the circumflex branch of the left coronary artery.

7. The prosthetic ring of any preceding claim, wherein a portion of the prosthetic ring opposite the gap: a) has an increased stiffness, b) has an increased thickness, and/or c) is formed from a stiffer material, compared to at least one other portion of the prosthetic ring.

8. The prosthetic ring of any preceding claim, wherein at least a portion of the prosthetic ring comprises multiple layers of material.

9. The prosthetic ring of any preceding claim, wherein at least a portion of the prosthetic ring opposite the gap has an increased number of layers of material.

10. The prosthetic ring of any preceding claim, wherein a portion of the prosthetic ring opposite the gap comprises a material with a flexural modulus which is higher than the material of the remainder of a circumference of the prosthetic ring.

11. The prosthetic ring of any preceding claim, wherein the prosthetic ring further comprises a suture permeable coating.

12. A prosthetic ring for repair of a mitral valve, having a substantially annular form or a part annular form, wherein the prosthetic ring occupies at least a part of a shape having:
a substantially straight base portion, configured to align with the aortic mitral curtain;
a central curved portion, arranged opposite the base portion, configured to align with the posterior leaflet of the mitral valve in the region of the middle scallop;
a first lateral curved portion, located between the first end of the central curved portion and the first end of the base portion, configured to align with the anterolateral commissure of the mitral valve;
a second lateral curved portion, located between the second end of the central curved portion and the second end of the base portion, configured to align with the posteromedial commissure of the mitral valve; and
wherein the prosthetic ring comprises a cut-able portion, located on the first lateral curved portion or the second lateral curved portion, which when removed, provides a gap in the circumference of the ring formed in the first lateral curved portion or the second lateral curved portion.

13. A method of preparing a prosthetic ring for repair of a mitral valve, comprising:
removing a portion of the ring;
wherein the prosthetic ring occupies at least a part of a shape having:
a substantially straight base portion, configured to align with the aortic mitral curtain;
a central curved portion, arranged opposite the base portion, configured to align with the posterior leaflet of the mitral valve in the region of the middle scallop;
a first lateral curved portion, located between the first end of the central curved portion and the first end of the base portion, configured to align with the anterolateral commissure of the mitral valve;
a second lateral curved portion, located between the second end of the central curved portion and the second end of the base portion, configured to align with the posteromedial commissure of the mitral valve; and
wherein removing the portion of the ring provides a gap in the circumference of the ring formed in the first lateral curved portion or the second lateral curved portion.

14. The method of preparing a prosthetic ring for repair of a mitral valve of claim 13, wherein the prosthetic ring has a substantially annular form or a part annular form, and further comprises:
a posterior ring part; and
an anterior ring part coupled to the posterior ring part at at least one coupling point on a transverse plane defining a maximum width of the prosthetic ring;
wherein removing the portion of the ring to provide a gap in the circumference of the ring comprises forming a gap located in the posterior ring part.

15. The method of preparing a prosthetic ring for repair of a mitral valve of claim 13, wherein removing the portion of the ring to provide a gap in the circumference of the ring comprises forming a gap in at least the portion of the prosthetic ring configured to align with the anterolateral commissure of the mitral valve.

16. The method of preparing a prosthetic ring for repair of a mitral valve of claim 13, wherein removing the portion of the ring to provide a gap in the circumference of the ring comprises forming a gap in at least the portion of the prosthetic ring configured to align with the anterior scallop of the mitral valve.

17. The method of preparing a prosthetic ring for repair of a mitral valve of claim 13, wherein removing the portion of the ring to provide a gap in the circumference of the ring comprises forming a gap in at least the portion of the prosthetic ring configured to align with the circumflex branch of the left coronary artery.

18. A prosthetic band for repair of a mitral valve, the prosthetic band forming at least part of a shape having:
a central curved portion, having a first radius of curvature, R₁; and
a first and second lateral curved portions, located either side of the central curved portion, wherein the first and second lateral curved portions have a second radius of curvature, R₂;
wherein R₂<R₁; and
a gap in the band, the gap extending over a base portion configured to align with the aortic mitral curtain and at least a portion of the first lateral curved portion or the second lateral curved portion.

19. A prosthetic band for repair of a mitral valve, having a substantially part annular form, wherein the prosthetic band occupies at least a part of a shape having:
a central curved portion, configured to align with the posterior leaflet of the mitral valve in the region of the middle scallop;
a first lateral curved portion, located at the first end of the central curved portion, configured to align with the anterolateral commissure of the mitral valve;
a second lateral curved portion, located at the second end of the central curved portion, configured to align with the posteromedial commissure of the mitral valve; and
a gap in the band, the gap extending over a base portion configured to align with the aortic mitral curtain and at least a portion of the first lateral curved portion or the second lateral curved portion.

20. The prosthetic band of claim 18 or 19, wherein the gap in the prosthetic band extends over at least the portion of the prosthetic band configured to align with the anterolateral commissure of the mitral valve.

21. The prosthetic band of any of claims 18 to 20, wherein the gap in the prosthetic band extends over at least the portion of the prosthetic band configured to align with the anterior scallop of the mitral valve.

22. The prosthetic band of any of claims 18 to 21, wherein the gap in the prosthetic band extends over at least the portion of the prosthetic band configured to align with the circumflex branch of the left coronary artery.

23. A prosthetic band for repair of a mitral valve, having a substantially part annular form, wherein the prosthetic band occupies at least a part of a shape having:
a central curved portion, configured to align with the posterior leaflet of the mitral valve in the region of the middle scallop;
a first lateral curved portion, located at the first end of the central curved portion, configured to align with the anterolateral commissure of the mitral valve;
a second lateral curved portion, located at the second end of the central curved portion, configured to align with the posteromedial commissure of the mitral valve; and
wherein the prosthetic band comprises a cut-able portion, located on the first lateral curved portion or the second lateral curved portion, which when removed, provides a gap in the band extending over the first lateral curved portion or the second lateral curved portion.

24. A method of preparing a prosthetic band for repair of a mitral valve, comprising:
removing a portion of the prosthetic band;
wherein the prosthetic band occupies at least a part of a shape having:
a central curved portion, configured to align with the posterior leaflet of the mitral valve in the region of the middle scallop;
a first lateral curved portion, located at the first end of the central curved portion, configured to align with the anterolateral commissure of the mitral valve;
a second lateral curved portion, located at the second end of the central curved portion, configured to align with the posteromedial commissure of the mitral valve; and
wherein removing the portion of the band provides a gap in the band extending over the first lateral curved portion or the second lateral curved portion.

25. The method of preparing a prosthetic band for repair of a mitral valve of claim 24, wherein removing the portion of the band to provide a gap in the band comprises forming a gap in at least the portion of the prosthetic band configured to align with the anterolateral commissure of the mitral valve.

26. The method of preparing a prosthetic band for repair of a mitral valve of claim 24, wherein removing the portion of the band to provide a gap in the band comprises forming a gap in at least the portion of the prosthetic band configured to align with the anterior scallop of the mitral valve.

27. The method of preparing a prosthetic band for repair of a mitral valve of claim 24, wherein removing the portion of the band to provide a gap in the band comprises forming a gap in at least the portion of the prosthetic band configured to align with the circumflex branch of the left coronary artery.

28. A method of repairing a mitral valve using a prosthetic ring of any of claims 1 to 12, or a prosthetic band of any of claims 18 to 23, comprising the steps of:
suturing the prosthetic ring, or the prosthetic band, to a mitral valve to remodel its shape.

29. A method of repairing a mitral valve comprising the steps of:
preparing a prosthetic ring according to the method of any of claims 13 to 17, or preparing a prosthetic band according to the method of any of claims 24 to 27; and
suturing the prosthetic ring, or the prosthetic band, to a mitral valve to remodel its shape.
